(19) United States
(12) Patent Application Publication
(10) Pub. No.: US 2016/0374704 A1
(43) Pub. Date: Dec. 29, 2016

(54) SYSTEMS AND METHODS FOR DISTRIBUTING SKIN PARTICLES

(71) Applicant: APPLIED TISSUE TECHNOLOGIES LLC, Hingham, MA (US)

(72) Inventors: Elof Eriksson, East Plainfield, NH (US); Michael Broomhead, Scituate, MA (US)

(21) Appl. No.: 15/196,422

(22) Filed: Jun. 29, 2016

Related U.S. Application Data
(60) Provisional application No. 62/185,944, filed on Jun. 29, 2015.

(51) Int. Cl. A61B 17/322; A61L 27/54; A61M 35/00; A61L 27/36

(52) U.S. Cl. CPC A61B 17/322; A61L 27/362; A61L 27/54; A61M 35/003; A61L 2430/34; A61B 2017/3225; A61B 2017/00969

(57) ABSTRACT

Systems and methods are provided to enable distribution of skin particles to a target site, for example, a wound site. Related kits are also provided.

### CROSS-REFERENCE TO RELATED APPLICATION

This application claims the benefit under 35 U.S.C. §119 of U.S. Provisional Patent Application Ser. No. 62/185,944, titled SYSTEMS AND METHODS FOR DISTRIBUTING SKIN PARTICLES, and filed Jun. 29, 2015, the entire disclosure of which is hereby incorporated herein by reference in its entirety for all purposes.

### BACKGROUND

Treatments of skin loss and reconstruction from trauma or burn injuries, for example skin wounds, skin defects, or scars are provided through specialized skin grafting and transplantation techniques. More specifically, split-thickness skin grafting and transplantation techniques are provided in order to improve wound treatments, for example for full-thickness wound healing.

### SUMMARY

In accordance with one or more aspects, a wound healing kit may comprise a source of a hydrogel, a mincing device configured to process harvested dermal tissue to form minced skin particles, a mixing device configured to mix the minced skin particles with the hydrogel to provide suspended skin particles, a distribution device for distributing the suspended skin particles at a target site, including a vessel for holding the suspended skin particles, and a delivery mechanism configured to cooperate with the vessel to distribute the suspended skin particles at the target site to promote wound healing, and instructions for operating at least one of the mincing device, the mixing device, and the distribution device.

In some embodiments, the target site is a wound or a bandage. In some embodiments, the mixing device comprises a syringe.

In some embodiments, the mixing device comprises two connectable syringes. In some embodiments, the syringe is connectable to a port of the vessel.

In some embodiments, the device is configured to distribute suspended skin particles have an average particle size of between about 0.1 mm and about 1.5 mm.

In some embodiments, the kit further comprises a bandage. In some embodiments, the kit further comprises a source of negative pressure to promote healing at the target site.

In some embodiments, the distribution device is configured to coat the suspended skin particles substantially evenly over the target site.

In some embodiments, the suspended skin particles include a therapeutic agent.

In some embodiments, the delivery mechanism comprises a plunger to deliver the suspended skin particles through application of pressure to the device.

In some embodiments, the distribution device is configured to distribute suspended skin particles to the target site at a pre-determined expansion ratio. In some embodiments, the expansion ratio is between about 1:10 to about 1:1000.

In accordance with one or more aspects, a method of distributing skin particles to a target site comprises providing skin particles having an average particle size of between about 0.1 mm and 1.5 mm, suspending the skin particles in a composition to provide a skin particle suspension, and actuating a device for delivering the suspended skin particles, the device comprising a vessel for holding the suspended skin particles, and a mechanism configured to cooperate with the vessel to distribute the suspended skin particles to the target site.

In some embodiments, providing the skin particles comprises providing a dermal tissue, cutting the dermal tissue in a first direction to provide a plurality of strips of the dermal tissue having a dimension T1, and cutting the plurality of dermal tissue strips in a second direction to provide a plurality of particles of the dermal tissue having a dimension T2, wherein each of the dimension T1 and the dimension T2 is approximately the same, and between about 0.1 mm and about 1.5 mm.

In some embodiments, actuating the device provides for dispersal of the skin particles at a pre-determined expansion ratio between about 1:10 to about 1:1000.

In some embodiments, the composition comprises a hydrogel.

In some embodiments, the target site is a wound or a bandage.

In some embodiments, the method further comprises maintaining a wet or moist environment at the target site.

In some embodiments, the method further comprises administering a therapeutic agent at the target site.

Still other aspects, embodiments, and advantages of these exemplary aspects and embodiments, are discussed in detail below. Any embodiment disclosed herein may be combined with any other embodiment in any manner consistent with at least one of the objects, aims, and needs disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

### BRIEF DESCRIPTION OF THE DRAWINGS

Certain illustrative features and examples are described below with reference to the accompanying figures in which:

FIG. 1 shows an embodiment of a vessel for holding suspended skin particles;

FIG. 2 shows an embodiment of a plunger configured to fit within the vessel;

FIG. 3 shows an embodiment of a luer cap connectable to the vessel;

FIG. 4 shows connectable syringes configured to mix skin particles and a suspension mixture;

FIG. 5 shows an embodiment of a device assembly comprising a vessel and a plunger;

FIG. 6 shows another embodiment of a device assembly comprising a vessel and a plunger;

FIG. 7 shows an embodiment of a plunger having one O-ring; and

FIGS. 8A, 8B, 8C, and 8D present data discussed in the accompanying Example.

It will be recognized by the person of ordinary skill in the art, given the benefit of this disclosure, that the dimensions, sizes, components, and views shown in the figures are for illustrative purposes. Other dimensions, representations, features, and components may also be included in the embodiments disclosed herein without departing from the scope of the description.

### DETAILED DESCRIPTION

The present disclosure provides for treatments of wounds. The treatments may provide for optimal healing of wounds, including accelerating healing processes, and improved aesthetics of the fully healed wounds.

The devices disclosed herein may be used in conjunction with, or be provided in a kit with the Xpansion® medical device and related methods commercially available from Applied Tissue Technologies LLC (Hingham, Mass.), as described in U.S. Pat. Nos. 7,625,384, 7,708,746, and 8,187,285, the contents of which are hereby incorporated herein by reference in their entireties for all purposes; or with PixelGrafting techniques disclosed in U.S. Provisional Patent Application Ser. No. 62/114,443, filed Feb. 10, 2015, the content of which is also hereby incorporated herein by reference in its entirety for all purposes.

Each year, approximately 500,000 burns require medical attention in the United States. Of these, 28% cover more than 10% of the patient's total body surface area. Such major burns are life threatening and require extensive treatment. The most common treatment is skin grafting. While skin grafts are typically successful, their maximum donor site expansion rate of 6-9 times means that patients with large burns often do not have enough donor skin to cover the entire wound. Additionally, grafting requires expensive surgical equipment and highly trained professionals, rendering the procedure difficult in low-resource settings.

An alternative to traditional skin grafting is mincing an autologous skin graft into small pieces and spreading it evenly over a wound. Mincing the skin releases wound healing signals which, combined with the increased total periphery per volume of skin, promotes more skin growth. This technique has shown an expansion rate of up to 100 times, greatly reducing the necessary size of donor sites. Current methods for spreading these pieces on a wound involve placing them individually with a spatula and forceps. While the existing minced skin technique allows for larger expansion ratios and smaller donor sites, it is slow and surgically cumbersome, and leads to uneven tissue distribution. The traditional technique is entirely serial: the wound bed is cleaned, the size of the necessary graft is measured, the donor site is harvested, and the procured graft is applied to the wound.

The present device spreads minced skin easily, quickly, and evenly over large target sites. In addition, the present method introduces the opportunity for parallel processing: while the surgeon debrides the wound bed, other health care providers in the operating room can harvest the graft, mince it, and prepare the pieces for spreading. Unlike traditional skin grafts where specifically the surgeon must typically size and subsequently suture or staple on the graft, due to the simplicity of minced skin grafts, any health care provider can procure, prepare, and apply the minced skin pieces. Because of its speed, ease of use, and hand-powered composition, the minced skin grafting procedure could be used in military settings and developing countries.

The treatments of the present disclosure may provide for treatments of full-thickness wounds. The full-thickness wounds may be caused by a major trauma or burn injury. The full-thickness wounds may increase the risk of mechanical and microbiological assaults. Skin grafting using split thickness skin grafts (STSGs) is an often used technology for treatment of full-thickness wounds. Wound healing may require a recapitulation, reforming, or restructuring of both the epidermal and dermal architecture including its interface, the basement membrane, to function normally. STSG may provide epidermal regeneration and may minimize wound contraction as compared to healing in non-transplanted full thickness wounds. In some instances, the STSG technique may be limited by availability of tissue, for example, donor site tissue. This may be the case with injuries that cover large areas of a subject. Meshed skin grafts may be the preferred choice of treatment in these instances. The meshed skin grafts may have an expansion ration of 1:1.5 to 1:6, which allows for limited donor tissue.

As used herein, expansion ratio is a ratio of the wound area (target site) to the donor site area. A higher expansion ratio, for example, a 1:100 expansion ratio, may be desired to minimize the trauma of the donor site, and to aid patients who have only a small amount of dermal tissue available for grafting purposes. Unfortunately, increased meshing ratio may result in poor aesthetics of the wound area. In these cases, healed wounds may have characteristics of a rough or uneven surface. In certain mesh grafting techniques, the healed wound may have a fish-net appearance.

In subjects having large wound areas requiring treatment, infection risks and healing problems increase due to re-use of the same area for donor tissue of the subject. For this reason, higher expansion ratios than previously used may be desirable.

Split-thickness skin graft (STSG) is a treatment of larger wounds from major trauma or burn injuries. In some instances, this treatment may be limited by the donor site availability in large burn injuries. Using a micrografting technique, an expansion ratio of 1:100 and wound healing comparable to STSG has been demonstrated in accordance with one or more embodiments. In accordance with one or more embodiments, both full and split thickness wounds may be treated.

The present disclosure relates to methods and materials for processing dermal tissue and treating wounds. The dermal tissue may be harvested from a subject. The process may involve cutting and mincing dermal tissue into particles suitable for application, for example, transplantation, into or to a wound of a subject.

The cutting and mincing of the dermal tissue into particles suitable for application may provide for skin particles, which may be prepared by the Xpansion® medical device and related methods, as described in U.S. Pat. Nos. 7,625,384, 7,708,746, and 8,187,285, the contents of which are hereby incorporated by reference in their entireties; or with PixelGrafting techniques disclosed in U.S. Provisional Patent Application Ser. No. 62/114,443, filed Feb. 10, 2015, the contents of which are also incorporated by reference in their entirety.

As used herein, the term “subject” is intended to include human, for example, a patient, and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the invention includes all vertebrates, for example, non-mammals (such as birds, for example, chickens; amphibians; reptiles) and mammals, such as non-human primates, domesticated, companion animals, and agriculturally useful animals, for example, sheep, dog, cat, cow, pig, rat, among others.

As used herein, micrografting comprises use of particles of dermal tissue having dimensions of greater than about 0.1 mm×0.1 mm up to about 5.0 mm×5.0 mm in a wound to provide tissue growth and healing. In some embodiments, micrografting comprises use of particles of dermal tissue having dimensions of greater than about 0.3 mm×0.3 mm up to about 5.0 mm×5.0 mm in a wound to provide tissue growth and healing. The thickness of the particles of dermal tissue may vary. In some instances, the thickness of the particles of dermal tissue may be about 0.35 mm. The thickness of the particles of dermal tissue may be between about 0.1 mm and about 1.0 mm, and may depend on the device used to provide the dermal tissue or the conditions of the wound. In some instances, the thickness of the particles may be between about 0.13 mm and about 0.84 mm. In other instances, the thickness of the particles may be between about 0.26 mm and about 0.45 mm.

As used herein, pixelgrafting comprises using particles of dermal tissue having dimensions of less than about 0.3 mm×0.3 mm in a wound to provide tissue growth and healing. In certain examples, pixelgrafting comprises using particles of dermal tissue having dimensions of less than about 0.1 mm×0.1 mm in a wound to provide tissue growth and healing. The thickness of the particles of dermal tissue may vary. In some instances, the thickness of the particles of dermal tissue may be about 0.35 mm. The thickness of the particles of dermal tissue may be between about 0.1 mm and about 1.0 mm, and may depend on the device used to provide the dermal tissue or the conditions of the wound. In some instances, the thickness of the particles may be between about 0.13 mm and about 0.84 mm. In other instances, the thickness of the particles may be between about 0.26 mm and about 0.45 mm.

While the particles of dermal tissue discussed herein may have dimensions and a thickness that may provide for cubelike particles, other shapes for the particles are contemplated by this disclosure, for example, rectangular, triangular or spherical.

Skin micrografts, 0.8×0 8 mm in size, may be provided from an autologous split-thickness skin graft (STSG) using a handheld mincing device as described in U.S. Pat. Nos. 7,625,384; 7,708,746; and 8,187,285, incorporated by reference herein in their entirety. The transplanted micrografts are able to regenerate epidermis and dermis in full-thickness porcine wounds in healthy as well as diabetic pigs. The wounds may be treated in a wet environment utilizing a polyurethane wound chamber that has been tested extensively in previous experiments. The wet environment enables the skin micrografts to migrate and proliferate independent of orientation.

Pixelgrafting, as described throughout this disclosure, minces donor skin to pieces measuring 0.3×0.3 mm or less. Without wishing to be bound by theory, by making the individual grafts smaller, the border length increases, thus increasing the regenerative capacity of the grafts. In addition, pixelgrafts could provide even larger expansion, i.e., larger expansion ratios may be used. The larger expansion areas may be beneficial in adequate or successful treatment of larger wound areas.

Accordingly, methods and materials are provided herein to treat a wound in a subject, for example, a full-thickness wound in a subject. In certain embodiments, a subject may be evaluated for suitability of a skin grafting procedure. This may include assessing the wound of the subject for suitability of a skin grafting procedure. The evaluation may also include determining an amount of the dermal tissue suitable for the wound, which may be determined, at least in part, on the size of the wound and the expansion ratio. Analgesics, antibiotics, anti-inflammatories, and other therapies may be provided to the subject at any point before, during, and after the treatment of the wound, as desired, or required.

The method may comprise providing a dermal tissue. The dermal tissue may be provided from a donor site of the subject, or from another source. The dermal tissue may be harvested, for example, from a donor site on the subject. The dermal tissue may then be treated to preserve its integrity, which may include disinfecting the dermal tissue and washing the dermal tissue in a buffer or culture media.

The dermal tissue may have specified dimensions. The dimensions may be specified based on one or more of an assessment of the size of the wound, an assessment of the condition of the wound, the location on the subject, for example a primarily visible location or a primarily non-visible location, a preselected expansion ratio, and the availability of donor tissue. The dermal tissue, or skin particles, may have a thickness, and a first dimension, T1, and a second dimension, T2. In certain examples, each of the dimension T1 and the dimension T2 is about 300 microns or less. In certain other examples, each of the dimension T1 and the dimension T2 is less than about 100 microns. It may be desirable to achieve the smallest dimensions for T1 and T2 that are achievable in order to optimize the healing process of the wound. The optimal healing process may provide for accelerated wound healing.

The method may comprise preparing a plurality of particles of dermal tissue, which may include cutting the dermal tissue into particles. The preparation of the particles of dermal tissue may be accomplished through the use of one or more cutting tools, and/or one or more cutting actions. For example the particles of dermal tissue may be prepared through use of a knife, blade, or cutting tool that may slice a dermal tissue into a plurality of dermal tissue strips, either simultaneously, or consecutively. The particles of dermal tissue may be further prepared through use of a knife, blade, or cutting tool that may cut the strips of dermal tissue into a plurality of dermal tissue particles, either simultaneously, or consecutively. The cutting actions may occur one or more times in order to achieve the desired dimensions of particles.

In at least some non-limiting embodiments, the particles may be between about 0.1 mm to about 1.5 mm, although smaller and larger particles may be contemplated. Once the plurality of particles of dermal tissue are prepared, they may be transplanted to the wound. The transplantation may occur without a concern for the orientation of the particles within the wound.

Transplantation, as referred to herein, may refer to providing prepared skin particles from a receptacle or surface to a target site, for example, a wound. Transplantation may involve the distribution or delivery of skin particles to a target site. The transplantation may include distribution of skin particles to a target site in an approximately even dispersal throughout and/or across the target site.

Transplantation may occur through use of a device. The device may provide for transplantation of the skin particles to a target site. For example, the target site may be a wound such as those described herein. In some embodiments, the target site may be a bandage or a film for placement over a wound, for instance, a large wound. For example, the target site may be a contact bandage having a size of about 20 cm×7.5 cm. In some embodiments, the bandage may be a mesh bandage, an antimicrobial bandage, or a waterproof bandage. The device may be configured to distribute the skin particles to the wound to provide full-thickness wound healing. The bandage may comprise adhesive or may be non-adhesive. In some embodiments, the bandage may absorb excess water and hydrogel from the wound. In some embodiments, the bandage may be a Tegaderm® Contact bandage. The device may be configured to distribute skin particles having an average particle size of between about 0.1 mm to about 1.5 mm. The device may be configured to distribute skin particles having an average particle size of about 0.1 mm. The device may be configured to distribute skin particles having an average particle size of about 1.0 mm. The device may be configured to distribute skin particles having an average particle size of about 1.5 mm.

The skin particles may be prepared as described herein and may be suspended in a composition, to provide a skin particle suspension. The skin particles may be distributed substantially evenly in the composition to form the suspension. In some embodiments, the skin particles may be suspended in water. In some embodiments, the skin particles may be suspended in cream or glue. In some embodiments, the skin particles may be suspended in a gel or a liquid. For example, the skin particles may be suspended in a hydrogel. The hydrogel may be any hydrogel that is sterile and indicated for an intended application, for example, first, second, and third degree burns. The hydrogel may be selected based on certain properties. For example, the hydrogel may be selected based on its viscosity. In some non-limiting embodiments, the hydrogel may be Carrasyn® gel commercially available from Carrington. The composition may aid in at least one of: the suspension of the particles; dispersion of the particles; delivery of the particles to the target site; adherence of the particles at the target site; and distribution, for example, approximately even distribution, or even distribution, of the particles throughout the target site. In embodiments, the composition may be a solution, gel, biologic adhesive, or non-biologic adhesive. The composition may be at least partially non-toxic to the dermal cells of the skin particles. The composition may aid in the maintenance of the viability of the skin particles. In embodiments, the solution may be a saline solution or a buffer solution that may have a pH suitable to maintain the viability of the dermal cells of the skin particles.

In embodiments, the composition, for example, solution, suspension, gel, or adhesive, may erode at the time of contact or within a short amount of time after contact of the particles in the target site. This may allow for nutrition to be based from the target site, for example, wound tissue to the skin particles.

The device may be a single use device that may be disposable, and optionally recyclable. The device may be packaged so that it is sterile prior to opening the package, and may allow for maintenance of a sterile environment upon disposal of the skin particles, or suspension comprising skin particles, into the device. It is contemplated that this device may be brought into an operating room, and may be loaded with the skin particles, or suspension comprising skin particles, in the operating room, or may be pre-loaded with the skin particles, prior to bringing into the operating room. The device may be biocompatible, sterile, and FDA approvable. In addition, the device may also deposit tissue quickly and efficiently, cover large wounds in a single graft, be intuitive to use, and not require external power. The device should be operable by one person, safe for use in an operating room, and must be ergonomic. In some embodiments, the device may be appropriate for use in the field, such as for military applications.

Embodiments of the disclosure may contemplate a multiple-use device that may be cleaned and sterilized for re-use.

In embodiments, the device for distributing skin particles may comprise a vessel for holding or receiving the skin particles, for example, suspended skin particles. The skin particles may be housed in the vessel in the composition. In embodiments, the skin particle suspension may be prepared prior to adding the skin particle suspension to the vessel such as with a syringe or a syringe system involving mated syringes. In other embodiments, the suspension may be prepared in the vessel. For example, a composition may be added to the vessel, and subsequently the skin particles may be added to the vessel, and the skin particles and composition may be mixed in the vessel to provide the suspension. In other examples, the skin particles may be added to the vessel, and subsequently the composition may be added to the vessel, and the composition and skin particles may be mixed in the vessel to provide the suspension.

The device may also comprise a mechanism configured to distribute the skin particles to the target site. This mechanism may cooperate with the vessel to deliver and/or distribute a suspension. Prevention of clogging is a design consideration in accordance with one or more embodiments. Various propulsion and/or delivery approaches may be implemented. The mechanism may allow for provision of a spray, mist or aerosol through actuation of a spray nozzle. The spray nozzle may allow for the suspension to be processed into small droplets to be delivered to the target site. The spraying may allow for even distribution, or approximately even distribution of skin particles or suspended skin particles throughout or across the target site. The spray nozzle may be of sufficient diameter to allow the passage of the skin particle suspension and to prevent clogging. For example, the spray nozzle may have a diameter larger than the diameter of the skin particle suspension.

In other embodiments, the mechanism may allow for dispersal of the suspension from the vessel by way of squeezing or applying pressure to the vessel, to push the suspension out of the device. For example, a spout may be provided to deliver the skin particles through application of pressure to the device.

In some embodiments, the device may comprise a vessel for holding the suspended skin particles, and a plunger for pushing the suspended skin particles through an opening in the vessel. The shape of the plunger may correspond to the shape of the vessel. The vessel may have at least one opening at one end to distribute the suspended skin particles. In some embodiments, one end of the vessel is entirely open. In other embodiments, the device may have more than one opening, or slot, at one end. The number and distribution of openings may impact distribution of suspended skin particles. The plunger may comprise an O-ring for creating a seal between the plunger and the vessel. The O-ring may also serve as a visual indicator of the amount of suspended skin particles distributed to the target site. In some embodiments, the vessel may comprise more than one O-ring. In some embodiments, the plunger has a rubber tip to create a seal between the plunger and the vessel.

The vessel may have an opening on another end, sized and configured to allow for passage of the body of the plunger. The opening may be defined by a lip, which prevents the base of the plunger from entering the vessel. This prevents the skin particle suspension from unintentionally seeping out of the vessel. The lip may be sized and configured to allow a user to support at least one finger on it.

The vessel may further comprise a cap with a luer fitting sized and configured to mate with a luer fitting on a syringe. The syringe may be configured to accept a pre-mixed skin particle suspension from a mixing device, such as a silicone bowl. In some embodiments, the syringe may be configured to accept skin particles and hydrogel for mixing prior to being transferred to the vessel. In some embodiments, a dual-syringe system is used to mix the skin particles and the hydrogel prior to introduction to the vessel. For example, 16 cc of a hydrogel may be loaded into a first 20 cc syringe. In some embodiments, the hydrogel may be pre-loaded into the first 20 cc syringe. Skin particles may be loaded into a second 20 cc syringe. The first and second syringes may be connected and the contents of one syringe may be transferred to the other syringe. For example, the hydrogel in the first syringe may be transferred to the second syringe to create a mixture. The new contents of the second syringe may then be transferred to the first syringe for more even mixing. This process may be repeated for a suitable time or number of transfers to ensure even mixing. For example, the contents may be transferred about 15 times at a rate of about one plunge every 2.5 seconds. The mixture or suspension may then be transferred via mating luer fittings from a syringe to the vessel. A cap may then be placed over the luer fitting on the vessel to prevent the mixture from leaking out.

The device may be manufactured by any reliable and economical means. For example, the device may be injection molded. In some embodiments, the vessel and the plunger may be manufactured as a two-part mold. In some embodiments, the vessel may be manufactured as a two-part mold. In this instance, the two parts of the mold may be secured together using a biocompatible adhesive, or by welding. The plunger may be manufactured and designed in accordance with standard syringe manufacturing procedures.

The device may be assembled and sold as part of a kit. For example, the kit may comprise the vessel, the one or more syringes, and the plunger. The kit may also comprise at least one luer cap. The kit may be sterilized before being used. The kit may be sterilized by any means suitable and effective for all kit components. For example, the components of the kit may be sterilized with ethylene oxide. In some embodiments, the kit may also contain instructions for using the device. In some embodiments, the kit may include a source of hydrogel for forming a suspension. The kit may in some embodiments include at least one bandage. The kit may include a source of negative pressure to promote healing. The kit may also include a mincing device for preparing or processing dermal tissue to form the minced skin particles for subsequent suspension.

In alternative embodiments, a brush or roller may be attached to the vessel, and may allow for distribution of the skin particle suspension from the vessel to the target site.

The composition used, such as the hydrogel, may provide for or aid in the dispersal of the skin particles to the target site, and may also aid in reducing or preventing clogging of the mechanism or device during use.

The device may be configured such that, upon delivery of the suspension comprising the skin particles, the device, for example, the mechanism, avoids, at least in part, shearing, or damaging, the dermal cells, or a device with a specific oncotic pressure.

It is estimated that each minced skin piece grows radially at a rate of about 0.5 mm/day. The device may be configured such that, upon delivery of the skin particle suspension, at least 95% of the skin particles epithelialize within two weeks. For example, about 98% of the particles may epithelialize within two weeks. In some embodiments, about 99% of the particles may epithelialize within two weeks. In some embodiments, 100% of the particles may epithelialize within two weeks.

The wound undergoing treatment may be maintained under preselected conditions to provide for healing as described above. For example, the wound undergoing treatment may be maintained in a negative pressure would chamber. The wound undergoing treatment, i.e., the treated wound, may be maintained in a wet environment. A wet environment may be established by enclosing the wound within the wound chamber that is composed of an impermeable membrane. The impermeable nature of the environment would yield no evaporation, and result in the creation of a wound microenvironment. Maintaining a wet or moist environment may be accomplished through use of a moist dressing that could be a wound chamber. The benefits of establishing a wet wound environment may include a better protected wound and the ability to deliver treatments topically, for example, analgesics, anti-inflammatories, antibiotics, antimicrobials, antifungals, or other treatments. The wound chamber may be provided through encapsulation of the wound in a structure that provides a barrier from the environment. This provides for a sterile, isolated environment. The wound chamber may allow for delivery of components to the wound in a sterile manner. For example, analgesics, antibiotics, anti-inflammatories, and other therapies may be provided to the wound. One or more of a growth supplement, a calcium-depleted serum, and an antibiotic may be provided to the wound chamber or to the plurality of particles of dermal tissue subsequent to transplanting the plurality of particles to the wound. Various therapeutic agents such as those described herein may also be included in the solution or suspension, such as a hydrogel suspension also including minced skin particles. The wound may be maintained in the wound chamber for a period of time to allow suitable treatment of the wound, for example, commencement of re-epithelialization or other properties that indicate wound healing. This period of time, for example, may be between about one day and about two weeks. More specifically, this period of time may be between about five and about seven days.

Through the methods and materials described in this disclosure, expansion ratios of greater than 1:2 may be used in order to treat a wound. For example, the expansion ratio may be 1:10, 1:50, 1:100, 1:200, 1:500, or 1:1000.

In certain embodiments, results may be achieved through use of the methods and materials of the disclosure that exceed results previously achieved. For example, the results achieved using the device of the present disclosure to distribute particles more evenly throughout the surface of the wound may provide for one or more of the following:
accelerated re-epithelialization,
accelerated incorporation of epidermis of the particles in neoepidermis,
decreased wound contraction,
increased neoepidermis thickness,
increased number of rete ridges per millimeter, and
improved expansion ratio,

as compared to a wound in which skin particles are distributed without the use of the device of the present disclosure.

### EXAMPLE

Skin Particle Distribution Device

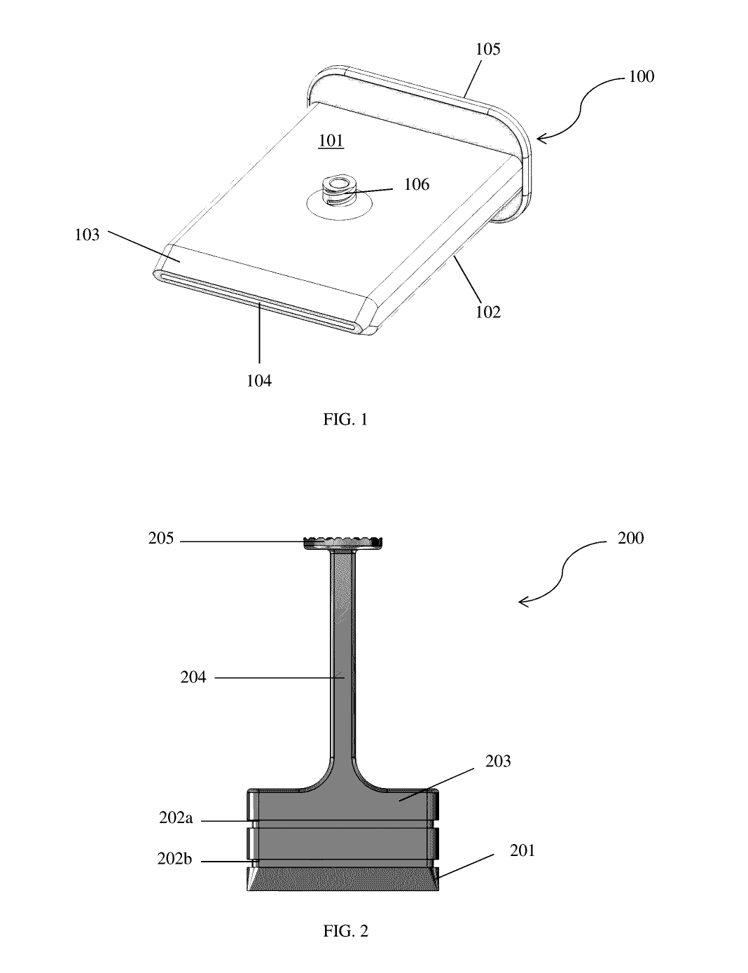

A device for distributing skin cells was created and tested. Referring to FIG. 1, the device comprises a vessel 100 having a body 101 for holding suspended skin particles. Suspended skin particles are transferred to body vessel 101 through the opening of luer fitting 106. Vessel body 101 has a height of 80 mm along length 102. Vessel body 101 further has an elongated opening 104 extending substantially entirely along first end 102. The output width of elongated opening 104 is 50 mm, and the output height is 1.05 mm. The opening is sized and configured to allow for the distribution of suspended skin particles from the vessel body 101. Vessel body 101 further comprised an opening (not shown) extending substantially entirely along the second end 106 of vessel body 101. Attached to vessel body 101 at second end 107 is raised lip 105. Raised lip 105 is sized and configured to prevent plunger head 205 (FIG. 2) from entering vessel body 101, and to allow a user to rest at least one finger on it during suspended skin particle application.

Plunger head 205 is at one end of plunger 200, and is sized and configured to not enter vessel body 101 and to promote ease of use. Plunger head 205 is connected to plunger stem 204 at one end. Plunger stem 204 is connected to plunger base 203 at another end. Plunger base 203 is sized and configured to fit within vessel body 101. Plunger base 203 has two grooves 202a and 202b, which are configured to hold O-rings. The grooves have a width of 2.0 mm and a depth of 1.8 mm. The O-ring depth is 1.8 mm. The O-rings are configured to provide a seal between the vessel body 101 and the plunger 200. At the other end of plunger base 203 is a plunger tip 201. Plunger tip 201 has a width of 48.5 mm Plunger 200 has a total height of 90 mm.

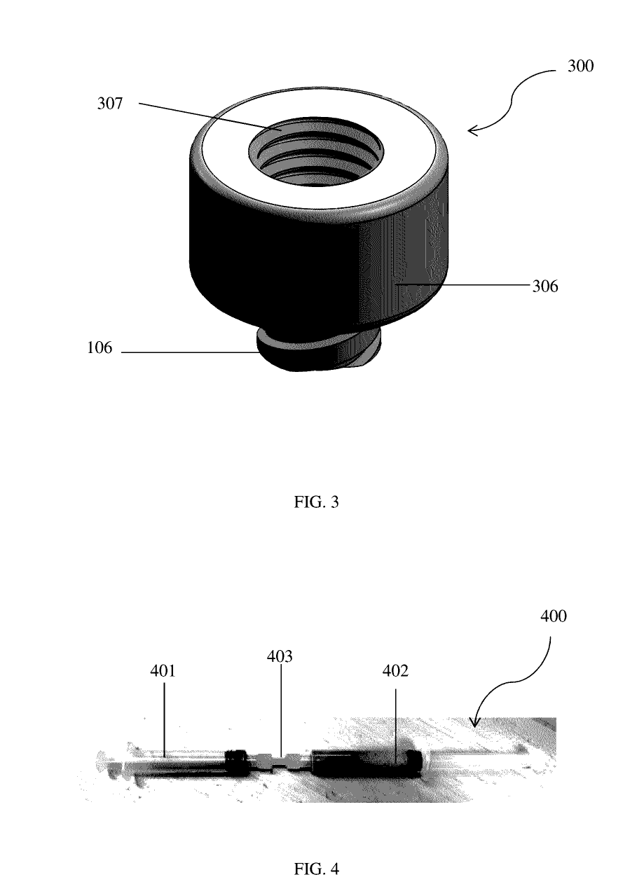

Referring to FIG. 3, a luer cap 300 is provided to fit over luer fitting 106. The threads 307 of luer cap 300 mate with the threads of luer fitting 106 to provide a seal from the environment at the luer fitting 106. The threads 307 have a 1.25 mm pitch, and are categorized as M9 thread.

Connectable 20 cc syringes 401, 402 are provided (FIG. 4) as mixing device 400. The syringes may be connected by connector 403. A first syringe 401 is configured to receive skin particles, and a second syringe 402 is configured to receive a hydrogel. A user mixes the skin particles and hydrogel into a suspended skin particle mixture by forcing the contents back and forth to each syringe. One end of connector 403 may be connectable to luer fitting 106 for transfer of the suspended skin particles from at least one of the first syringe 401 and the second syringe 402 to the vessel body 101.

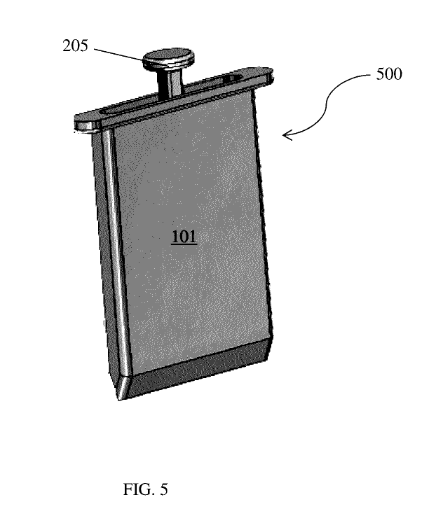

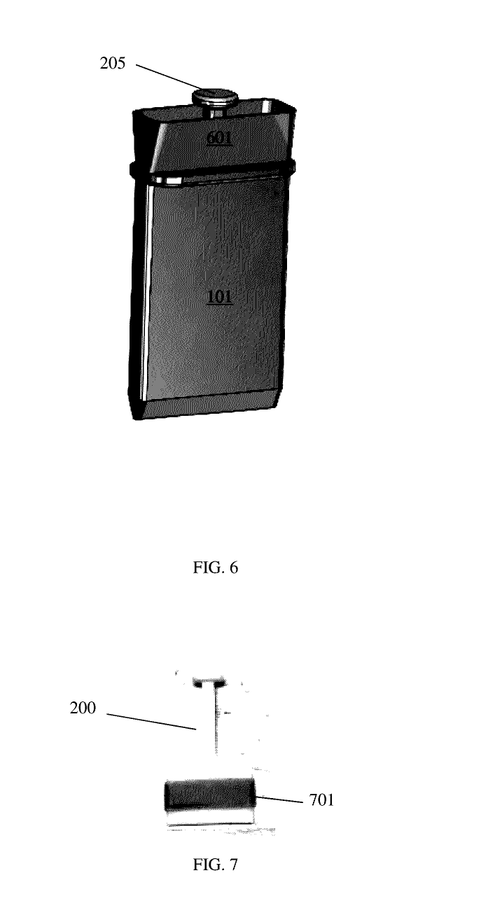

FIG. 5 illustrates the device assembly 500 comprising vessel body 101 and plunger 200. As shown in FIG. 5, plunger head 205 does not enter vessel body 101.

Application

About 4 cm² of apple skin, as a replacement for human skin, was minced into 0.8 mm×0.8 mm particles with the Xpansion® medical device. The skin pieces were inserted into a first 20 cc syringe with the plunger removed, and the plunger was subsequently inserted to the 1 mm mark of the syringe. Next, 16 cc of hydrogel was squeezed into a second 20 cc syringe. The first and the second syringes were connected to each other, and the plungers were actuated to push the suspended skin particle mixture back and forth 15 times for 2.5 s each plunge.

The suspended skin particle mixture was transferred to the vessel body 101 through luer fitting 106. Luer cap 300 was placed on luer fitting 106 to seal vessel body 101 from the environment.

A user actuated the plunger 200 within vessel body 101 at a sufficient speed and angle to distribute the suspended skin particles onto a 20 cm×7.5 cm Tegaderm® bandage commercially available from 3M. The bandage was split into four separate section, and about 4 mL of suspended skin particles were distributed per section. This process was repeated eight times.

Results

A picture was taken after each application of suspended skin particles to a bandage. Each time, the bandage was transferred skin side down to a foam board, and another picture was taken. The distributions before and after transfer were analyzed using a Matlab program to determine the distance between every particle and its closest particle. This information determined the percent epithelialization as a function of time.

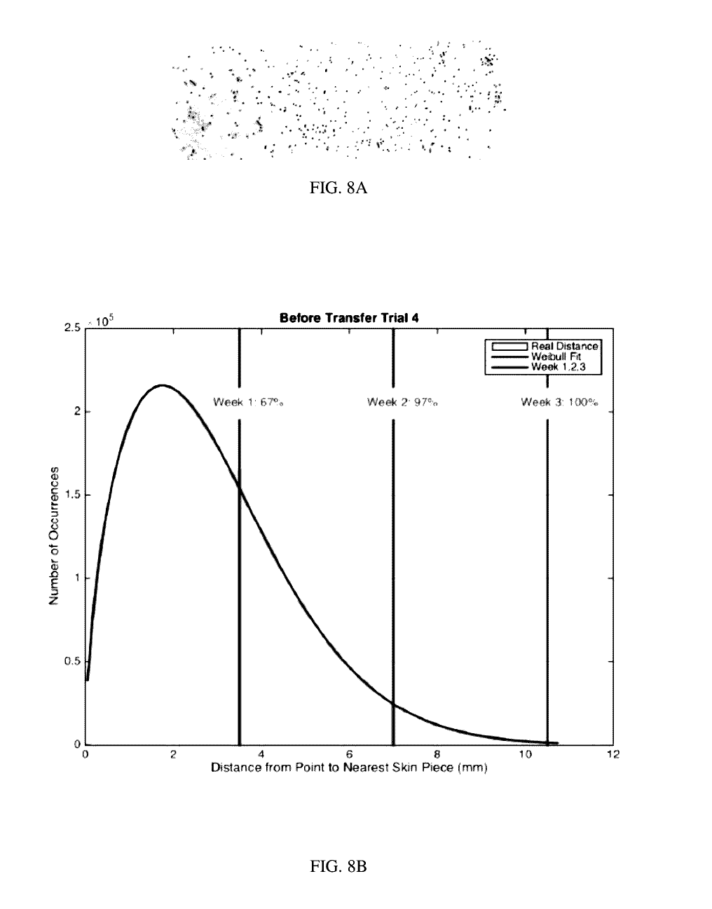

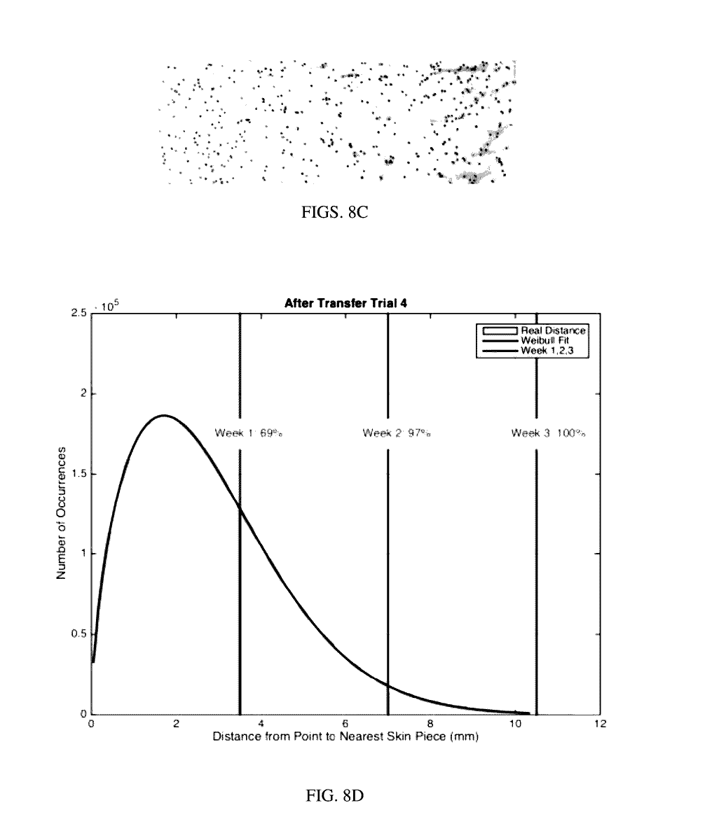

All eight trials showed satisfactory distribution both before and after transferring the bandage, placing it skin side down. FIGS. 8A and 8B provide representative data collected during one of the trials prior to transferring the bandage. FIGS. 8C and 8D provide representative data collected during the same trial as that of FIGS. 8A and 8B but after transferring the bandage. The graphs (FIGS. 8B and 8D) are Weibull curves which fit the distribution of distances from points on the wound to the nearest skin piece (FIGS. 8A and 8C). To determine the percentage area of the wound epithelialized after a certain number of days, the cumulative distribution function is used. The area under the curve equals the percentage of area healed and follows Equation (1).

F(x, k, λ)=1−e(−x/λ)^{k} (1)

All pixels within 3.5 mm (0.5 mm/day*5 days) of the nearest skin piece epithelialize within the first week, pixels within 7 mm epithelialize within two weeks, and pixels within 10.5 mm epithelialize within three weeks. Each trial showed a predicted 95% or higher epithelialization at two weeks before and after transferring the bandage.

Alternate embodiments of device assembly 500 are contemplated. For example, in one embodiment, vessel body 101 may further comprise a hood 601, positioned above raised lip 105. This hood may provide for a greater area on which a user may rest at least one finger during operation.

In other embodiments, the plunger 200 may comprise only one O-ring 701. The O-ring may be sized and configured to create a seal between vessel body 101 and plunger 200.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure, and are intended to be within the scope of the invention. Accordingly, the foregoing description and drawings are by way of example only.

What is claimed is:

1. A wound healing kit, comprising:
a source of a hydrogel;
a mincing device configured to process harvested dermal tissue to form minced skin particles;
a mixing device configured to mix the minced skin particles with the hydrogel to provide suspended skin particles;
a distribution device for distributing the suspended skin particles at a target site, comprising:
a vessel for holding the suspended skin particles; and
a delivery mechanism configured to cooperate with the vessel to distribute the suspended skin particles at the target site to promote wound healing; and
instructions for operating at least one of the mincing device, the mixing device, and the distribution device.

2. The kit of claim 1, wherein the target site is a wound or a bandage.

3. The kit of claim 1, wherein the mixing device comprises a syringe.

4. The kit of claim 3, wherein the mixing device comprises two connectable syringes.

5. The kit of claim 3, wherein the syringe is connectable to a port of the vessel.

6. The kit of claim 1, wherein the device is configured to distribute suspended skin particles have an average particle size of between about 0.1 mm and about 1.5 mm.

7. The kit of claim 1, further comprising a bandage.

8. The kit of claim 1, further comprising a source of negative pressure to promote healing at the target site.

9. The kit of claim 1, wherein the distribution device is configured to coat the suspended skin particles substantially evenly over the target site.

10. The kit of claim 1, wherein the suspended skin particles includes a therapeutic agent.

11. The kit of claim 1, wherein the delivery mechanism comprises a plunger to deliver the suspended skin particles through application of pressure to the device.

12. The kit of claim 1, wherein the distribution device is configured to distribute suspended skin particles to the target site at a pre-determined expansion ratio.

13. The kit of claim 12, wherein the expansion ratio is between about 1:10 to about 1:1000.

14. A method of distributing skin particles to a target site, comprising:
providing skin particles having an average particle size of between about 0.1 mm and 1.5 mm;
suspending the skin particles in a composition to provide a skin particle suspension; and
actuating a device for delivering the suspended skin particles, the device comprising:
a vessel for holding the suspended skin particles; and
a mechanism configured to cooperate with the vessel to distribute the suspended skin particles to the target site.

15. The method of claim 14, wherein providing the skin particles comprises:
providing a dermal tissue;
cutting the dermal tissue in a first direction to provide a plurality of strips of the dermal tissue having a dimension T1; and
cutting the plurality of dermal tissue strips in a second direction to provide a plurality of particles of the dermal tissue having a dimension T2, wherein each of the dimension T1 and the dimension T2 is approximately the same, and between about 0.1 mm and about 1.5 mm.

16. The method of claim 14, wherein actuating the device provides for dispersal of the skin particles at a pre-determined expansion ratio between about 1:10 to about 1:1000.

17. The method of claim 14, wherein the composition comprises a hydrogel.

18. The method of claim 14, wherein the target site is a wound or a bandage.

19. The method of claim 14, further comprising maintaining a wet or moist environment at the target site.

20. The method of claim 14, further comprising administering a therapeutic agent at the target site.
